(19) United States
(12) Patent Application Publication
(10) Pub. No.: US 2005/0142586 A1
(43) Pub. Date: Jun. 30, 2005

(54) Measuring enzymatic activity

(76) Inventor: Herbert Mason Hedberg, North Attleboro, MA (US)

Correspondence Address: FISH & RICHARDSON PC, 225 FRANKLIN ST, BOSTON, MA, 02110, US

(21) Appl. No.: 10/957,851

(22) Filed: Oct. 4, 2004

Related U.S. Application Data
(60) Provisional application No. 60/525,538, filed on Nov. 26, 2003.

(51) Int. Cl. C12Q 1/68; C12P 19/34

(52) U.S. Cl. 435/6; 435/91.2

(57) ABSTRACT

Disclosed is a process for measuring activity of an enzyme using a dialysis device comprised of a reaction chamber containing a substrate, a reservoir chamber containing the substrate, and a membrane separating the reaction chamber and the reservoir chamber. The process includes exposing the substrate in the reaction chamber to an enzyme and a test substance, where the membrane inhibits passage of the enzyme and the test substance, and detecting a change in an amount of the substrate in the reservoir chamber after the substrate has been exposed to the enzyme and the test substance for a time. A reaction that occurs in the reaction chamber between the substrate and the enzyme affects the amount of the substrate that passes through the membrane from the reservoir chamber to the reaction chamber. The test substance affects the reaction.

### CROSS-REFERENCE TO RELATED APPLICATION

This application claims the benefit of U.S. Provisional Application No. 60/525,538, filed on Oct. 12, 2003, entitled “Method And Device For Measuring Telomerase And Other Enzyme Activity”, the contents of which are hereby incorporated by reference into this application as if set forth herein in full.

### TECHNICAL FIELD

This patent application relates generally to measuring enzymatic activity and, more particularly, to using a dialysis-based assay to measure the activity of telomerase.

### BACKGROUND

During replication of a linear strand of DNA, there is not enough room to form the last Okizaki fragment on the 3′ end of the lagging strand, causing some genetic material to be lost. With several replications, a cell would eventually become non-viable. Telomerase, a reverse-transcriptase, extends the end of a chromosome using an RNA molecule as a template, forming a long, single-stranded DNA molecule, comprised of a repeating sequence: (5′-TTAGGG). This extension allows another Okizaki fragment to be added, and no genetic information is lost. The remaining single-stranded DNA, the telomere, folds-up with telomere binding proteins to physically protect the end of the chromosome.

Telomerase is comprised of a protein and an RNA subunit, as shown in FIG. 1. The protein is called telomerase reverse transcriptase (hTERT), and the RNA subunit is called telomerase RNA (hTR). Most immortal eukaryotic cell lines, such as fungi and yeast, contain active telomerase, which maintains the telomere for infinite cell divisions. Human fetal cells also have active telomerase which builds a telomere of sufficient size to allow about fifty replications during the normal lifespan of the individual. However, before birth, telomerase becomes inactive. The chromosomes begin life with long telomeres, which become progressively shorter with each cell division. This continues until the telomeres reach a critical length, called the Hayflick limit, whereafter DNA replication, and therefore cell division, stops. Active telomerase is also found in 80% of cancer cell types and enables uncontrolled replication of cancerous cells much like that found in eukaryotic cells.

Because cancerous cells contain the only active telomerase in a human, telomerase inhibitors target almost exclusively cancerous tumors. By inhibiting telomerase, cancerous cells would continue to divide, but surpass the Hayflick limit, lose genetic material and become non-viable. This has been demonstrated using antisense hTR as a telomerase inhibitor in mouse tumor cells. Therefore, much current research is focused on identifying telomerase inhibitors for potential use as anti-cancer agents.

A currently-used assay, the telomerase repeat amplification protocol (TRAP), involves a complex process, which allows telomerase from cell lysate to extend primers of telomeric sequence and then amplify the sequence with PCR. The results are analyzed by radioactive methods or by a gel. The entire process can take up to two days to complete.

### SUMMARY

In general, in one aspect, the invention is directed to a method that includes exposing an enzyme to a substrate and a test substance in a first chamber, and detecting an effect on the substrate in a second chamber following exposure of the enzyme to the substrate and the test substance in the first chamber. This aspect of the invention may also include one or more of the following features.

The enzyme may be telomerase and the substrate may include nucleotides.

Detecting the effect on the substrate may include determining a change in an amount of light absorbed by the substrate in the second chamber following exposure. The light may include ultraviolet light and/or laser light.

Exposing the enzyme to the substrate in the first chamber may cause a reaction that reduces an amount of free substrate in the first chamber. This may result in the substrate in the second chamber migrating to first chamber in response to a reduction in the amount of free substrate in the first chamber. The reaction may include formation of single-stranded DNA using the substrate and the enzyme. The test substance may affect the reaction. The effect on the reaction may be inhibiting or promoting the reaction.

In general, in another aspect, the invention is directed to a method of measuring activity of an enzyme using a dialysis device comprised of a reaction chamber containing a substrate, a reservoir chamber containing the substrate, and a membrane separating the reaction chamber and the reservoir chamber, where the membrane allows passage of the substrate. The method includes exposing the substrate in the reaction chamber to an enzyme and a test substance, the membrane inhibiting passage of the enzyme and the test substance, and detecting a change in an amount of the substrate in the reservoir chamber after the substrate has been exposed to the enzyme and the test substance for a time. The amount of the substrate in the reservoir chamber corresponds to an amount of the substrate that has passed through the membrane from the reservoir chamber to the reaction chamber. A reaction that occurs in the reaction chamber between the substrate and the enzyme affects the amount of the substrate that passes through the membrane from the reservoir chamber to the reaction chamber. The test substance affects the reaction.

The above aspect may also include one or more of the following features. The test substance may inhibit the reaction. The enzyme may include at least one of: telomerase, DNA polymerase, RNA polymerase, RNA ligase, ribosomes, and starch synthase. The substrate may include nucleotides, and the test substance may include a telomerase inhibitor. Detecting the change in the amount of the substrate may include measuring a change in absorption of ultraviolet light by the substrate in the reservoir chamber.

In general, in another aspect, the invention is directed to an apparatus that includes a reservoir chamber that contains a substrate; a reaction chamber that contains the substrate, an enzyme, and a test substance; and a dialysis membrane that separates the reservoir chamber and the reaction chamber. The dialysis membrane permits passage of the substrate and inhibits passage of the enzyme and the test substance. The test substance has an effect on a reaction between the substrate and the enzyme. The reaction influences an amount of the substrate that migrates from the reservoir chamber, through the dialysis membrane, to the reaction chamber. This aspect may also include one or more of the following features.

The test substance may influence the reaction by inhibiting the reaction or promoting the reaction. The enzyme may include at least one of: telomerase, DNA polymerase, RNA polymerase, RNA ligase, ribosomes, and starch synthase. The substrate may include nucleotides, and the test substance may include a telomerase inhibitor.

The apparatus according to the invention may include an ultraviolet light source that exposes at least the reservoir chamber to ultraviolet light. An amount of ultraviolet light absorbed by the substrate in the reservoir chamber corresponds to the effect the test substance has on the reaction. The reaction may include formation of single-stranded DNA using the substrate and the enzyme. The reservoir chamber may be a low volume equilibrium dialysis chamber.

### DESCRIPTION OF THE DRAWINGS

FIG. 1 is a diagram showing the structure of telomerase.

FIG. 2 is a diagram of a dialysis device that is used in an assay to detect activity of telomerase in the presence of a substrate and a test substance.

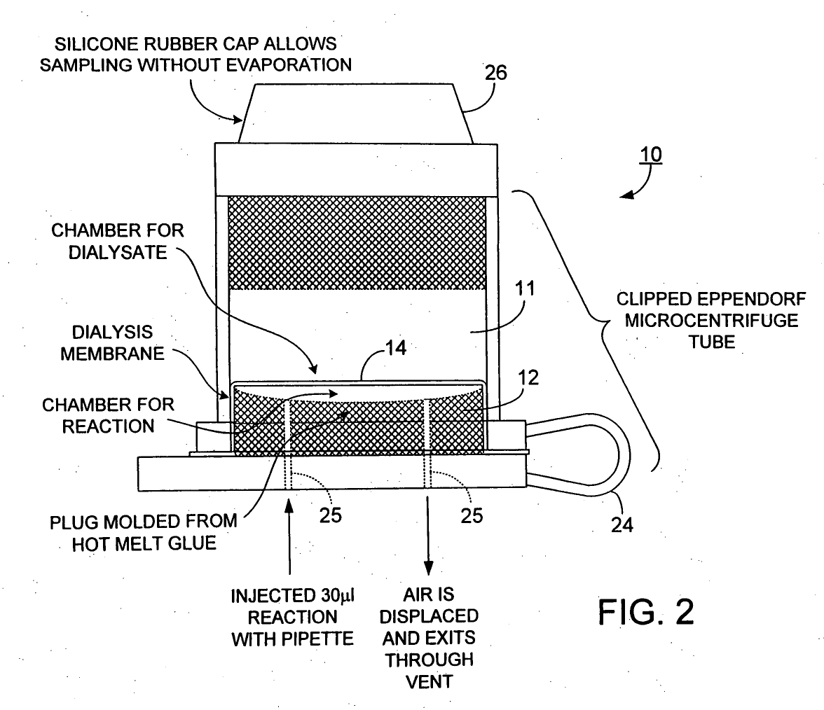

FIG. 3 is graph showing how ultraviolet (UV) absorbance of dialysate in a reservoir chamber of the dialysis device decreases over time in response to a reaction between telomerase and substrate occurring in a reaction chamber of the dialysis device.

FIG. 4 shows the reservoir chamber, the reaction chamber, and a graph indicating a decrease in UV absorbance of the dialysate in the reservoir chamber as a result of the reaction between telomerase and the substrate in the reaction chamber.

FIG. 5 is a graph showing different decreases in UV absorbance for different telomerase inhibitors in the reaction chamber.

FIG. 6 is a graph indicating a decrease in UV absorbance of the dialysate in the reservoir chamber as a result of a reaction in the reaction chamber.

FIG. 7 shows the reservoir chamber, the reaction chamber, and a graph indicating a decrease in enzymatic activity in the reaction chamber.

FIG. 8 shows the reservoir chamber, the reaction chamber, and a graph indicating an increase in enzymatic activity in the reaction chamber.

Like reference numerals in different figures indicate like elements.

### DETAILED DESCRIPTION

Described herein is a telomerase activity assay, which measures the effect of a test substance (e.g., a telomerase inhibitor) on a reaction between telomerase and a substrate (e.g., nucleotides). The assay can be used to determine the effectiveness of telomerase inhibitors. In one embodiment, the assay measures the rate at which telomerase assembles nucleotides into single-stranded DNA in the presence of a natural compound. This rate is then used to determine the effectiveness of this molecule as a telomerase inhibitor.

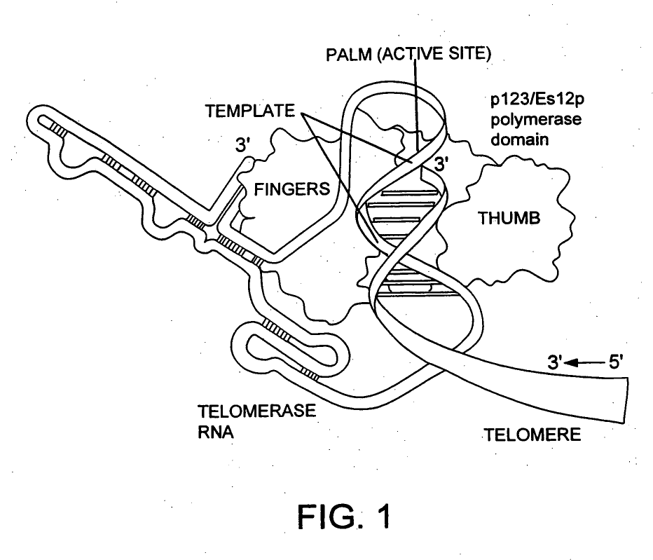

One way of measuring the rate at which telomerase assembles nucleotides into single-stranded DNA employs a dialysis device, such as that shown in FIG. 1. In this embodiment, dialysis device 10 is an equilibrium dialysis chamber.

Dialysis device 10 contains at least two chambers: a reservoir chamber 11 and a reaction chamber 12. A dialysis membrane 14 separates the two chambers. Reservoir chamber 11 contains the dialysate with the same concentration of small molecule substrate (or simply “substrate”), such as nucleotides, as the reaction. Reaction chamber 12 contains the enzymatic reaction: telomerase, the substrate, and a test substance, such as a telomerase inhibitor. A telomerase inhibitor inhibits (e.g., reduces or prevents) an enzymatic reaction that causes telomerase to assemble the substrate into single-stranded DNA.

Reservoir chamber 11 provides a mechanism for periodically sampling the dialysate without removing, from the reaction chamber, any large molecules, such as enzymes (e.g., telomerase), primers or products. For example, the reservoir chamber may provide an opening into which a pipette can be inserted. In this configuration, the reservoir chamber also reduces evaporation of the dialysate, which would otherwise induce concentration changes not due to nucleotide assembly by telomerase.

Dialysis membrane 14 isolates the enzymatic reaction in reaction chamber 12 from reservoir chamber 11. More specifically, dialysis membrane 14 allows passage of small molecules, such as substrate, but prevents passage of larger molecules, such as telomerase, single-stranded DNA, and a test substance. In this example, telomerase assembles into larger molecules in reaction chamber 12. In the presence of dialysis membrane 14, this reaction causes the substrate to diffuse from reservoir chamber 11 into reaction chamber 12. Thus, the amount of small molecule substrates in reservoir chamber 11 depends upon the reaction between telomerase and the substrate in the reaction chamber. The rate of change in concentration of substrate in the dialysate can thus be used to quantify enzyme activity.

More specifically, nucleotides in the dialysate pass across membrane 14 from reservoir chamber 11 into reaction chamber 12 as telomerase assembles nucleotides into DNA (called “the telomerase reaction”). That is, free nucleotides move from an area of high concentration (reservoir chamber 11) to an area of increasingly lower concentration (reaction chamber 12). The lower concentration of free nucleotides in reaction chamber 12 results from telomerase combining with the nucleotides in reaction chamber 12 to form the single-stranded DNA. Because the nucleotides absorb UV (ultraviolet) light, the dialysate's UV absorbance decreases as the telomerase reaction progresses (i.e., as the nucleotides move from reservoir chamber 11 to reaction chamber 12).

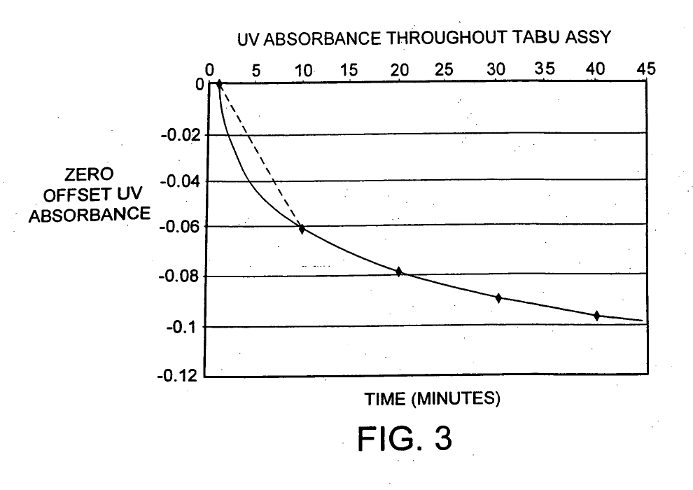

The assay described herein detects the foregoing telomerase reaction by detecting changes in nucleotide concentration in reservoir chamber 11 as telomerase synthesizes telomeric (5′-TTAGGG) repeats. FIG. 3 shows that the UV absorbance of the dialysate (i.e., the solution in reservoir chamber 11 containing substrate) decreases exponentially over time. This decrease in UV absorbance indicates that nucleotides have passed from reservoir chamber 11 to reaction chamber 12 (which, in turn, indicates that the telomerase in reaction chamber 12 is reacting with the nucleotides to form single-stranded DNA). If there is little or no decrease in UV absorbance, this indicates that few or no nucleotides have passed from reservoir chamber 11 to reaction chamber 12 (which, in turn, indicates that the telomerase inhibitor in reaction chamber 12 is inhibiting the reaction between telomerase and the nucleotides in reaction chamber 12). In FIG. 3, absorbance of nucleotides was measured every ten minutes and an exponential decrease occurred.

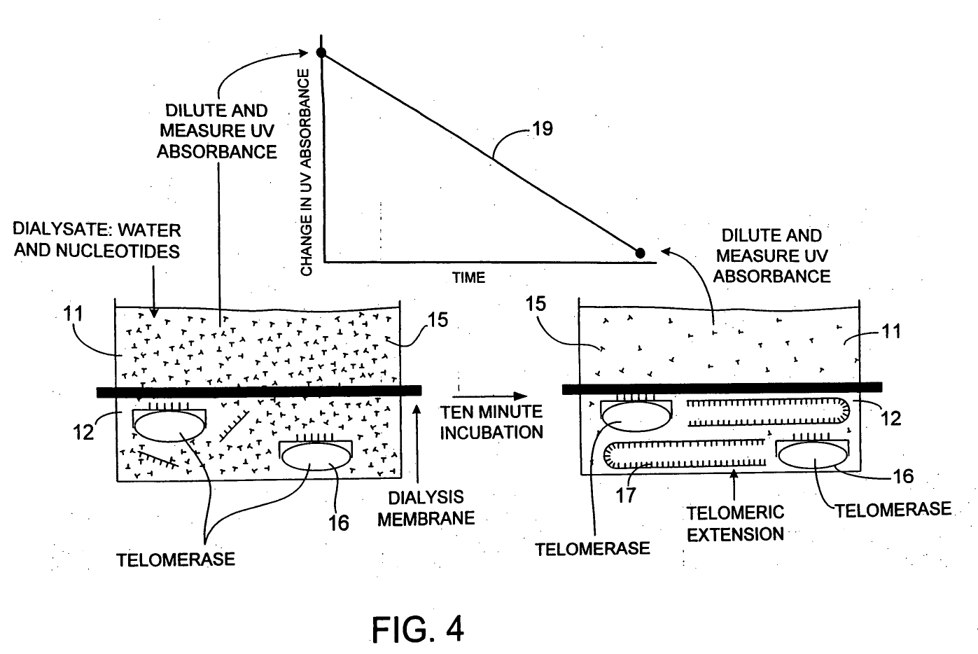

FIG. 4 shows another graph that illustrates the change in UV absorbance over time as a result of enzymatic activity. More specifically, FIG. 4 shows reservoir chamber 11 containing substrate 15, and reaction chamber 12 containing substrate 15 and telomerase 16. As shown, during an incubation period of ten minutes, telomerase reacts with the substrate in reaction chamber 12 to form single-stranded DNA 17. This causes substrate 15 to diffuse (migrate) from reservoir chamber 11 into reaction chamber 12, resulting in a lower concentration of substrate in reservoir chamber 11. This lower concentration results in less UV absorption from reservoir chamber 11, as shown in graph 19.

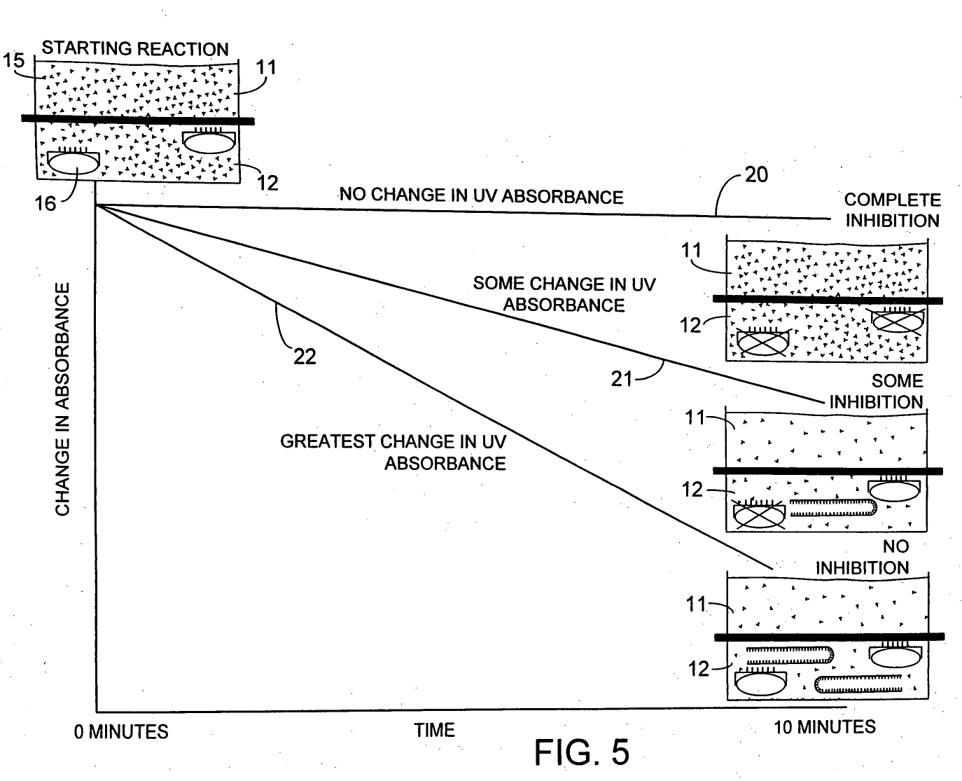

FIG. 5 shows how UV absorbance changes for different levels of enzymatic activity. More specifically, line 20 indicates no change in UV absorption in reservoir chamber 11. This means that the telomerase inhibitor in reaction chamber 12 has completely inhibited the reaction between telomerase and the nucleotides. Line 21 indicates some change in UV absorption in reservoir chamber 11. This means that the telomerase inhibitor in reaction chamber 12 has at least partly inhibited the reaction between telomerase and the nucleotides. Line 22 indicates a greater change in UV absorption in reservoir chamber 11. This means that the telomerase inhibitor in reaction chamber 12 has had less of an effect on the reaction between telomerase and the nucleotides. A large amount of change in UV absorption can indicate that the inhibitor has had little or no effect.

In one embodiment, the telomerase reaction includes a telomerase reaction buffer, concentrated telomerase, telomeric primer (5′-TTAGGG), nucleotides, and any compounds (i.e., test substance) to be tested as telomerase inhibitors. The dialysate is comprised of water and an equal concentration of nucleotides to that of the reaction. The membrane prevents the large molecules of the reaction (the telomeric primer and telomerase enzyme) from passing into the dialysate in the reservoir chamber. As telomerase synthesizes, telomeric repeats are formed from nucleotides, nucleotide concentration decreases in the reaction chamber, and nucleotides from the reservoir chamber pass through the membrane into the reaction chamber to maintain concentration equilibrium among the solutes. Periodic sampling from the dialysate chamber for nucleotide quantification has little or no effect upon the reaction proceeding on the reaction side of the dialysis membrane.

Obtaining a rate of nucleotide consumption provides sufficient data with which to rank telomerase inhibitor compounds in a screening campaign. In this embodiment, a unique low volume equilibrium dialysis chamber provides an isolated reservoir of free nucleotides from which measurements could be made without interference from reactants or products. As noted, FIG. 1 shows an example of a dialysis device that may be used.

In the embodiment of FIG. 1, reaction chamber 12 and reservoir chamber 11 are constructed as follows: clip the top 0.5 inches off an Eppendorff 1.5 ml microcentrifuge tube 24; fill the cap of the tube with hot melt glue; press the bottom of a Spectronic-20 glass cuvette into the glue; wipe away the excess glue; and carefully remove the cuvette with a twist so the glue remains in the shape of the bottom of the cuvette. This leaves a reaction chamber 12 of approximately 30 μl. With a 1 mm drill, bore two holes 25 through the cap and glue along the diameter of the cap about 0.25 inches apart. This allows the reaction to be injected into the chamber and the air to be displaced. Next, push a silicon-rubber septum 26 (Beckman MDQ sample vial closure) into the clipped end of the tube In one embodiment, the assay includes mixing a reaction. The components include 36 μl 2× telomerase reaction buffer, 8 μl 12M dNTP, 3 μl telomeric primer, 0.5 μg/μl, 2 μl dH₂O, and 0.5 μl telomerase. The assay also includes mixing the dialysate. The dialysate includes 8 μl 12M dNTP and 42 μl dH₂O. A circular, 0.5 inch diameter 2 kDa cellulose dialysis membrane is placed across the reaction chamber, and a cap on the chamber is closed. The cap contains a hole, into which 30 μl of reaction is injected. The cap is wrapped with Parafilm, and the end of the chamber is kept down. 50 μl dialysate is injected through a silicon-rubber cap on top of the dialysis membrane into the reservoir chamber. To measure nucleotide concentration in the dialysate, remove 2 μl, dilute into 98 μl dH₂O, and measure UV absorbance. Periodic measurements of the dialysate in the reservoir chamber may be made in order to quantify telomerase activity in the reaction chamber.

The synthesis rate in the reaction chamber (telomerase activity) is inferred by measuring a decrease in nucleotide concentration over time of the dialysate in the reservoir chamber. A perforated silicone septum on the top of the chamber can be used to prevent or reduce erroneous concentration changes due to evaporation.

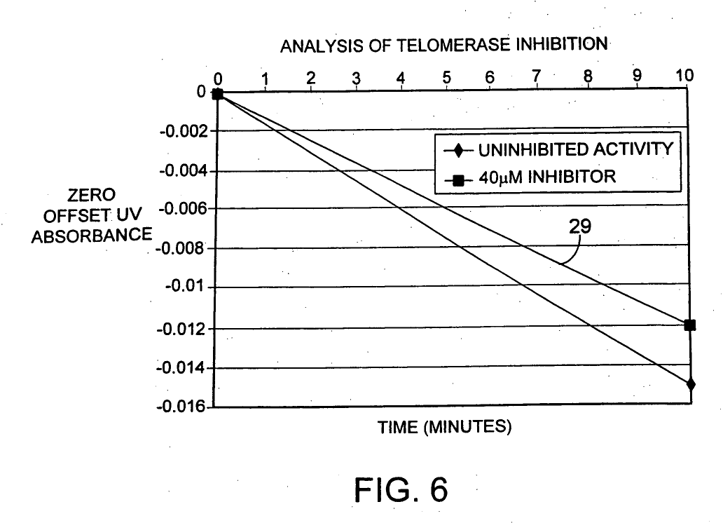

FIG. 6 shows that it is possible to detect the difference in activity between inhibited and uninhibited reactions in as little as ten minutes. UV measurements are taken at the start of each of two reactions and again after a ten-minute incubation period. The resulting graph shows two lines, of which line 29 (with the smaller slope) corresponds to the reaction with lower telomerase activity.

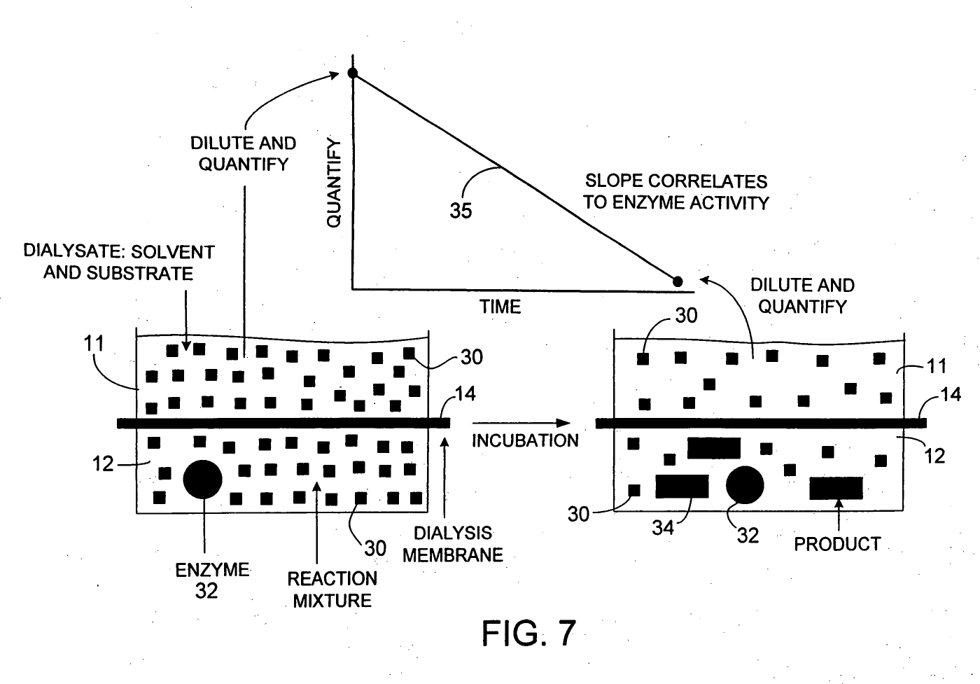

The assay described herein is not limited to use with measuring telomerase activity. Instead, the assay can be used to measure the activity of any type of enzyme. For example, the assay could be used to measure the activity any enzyme that constructs a large product from a smaller substrate. Examples of such enzymes (aside from telomerase) include, but are not limited to, DNA polymerase, RNA polymerase, DNA ligase, ribosomes, and starch synthase. FIG. 7 shows an illustration of how the assay can be employed in this context. The principle of FIG. 7 is the same as that of FIG. 3. More specifically, an enzymatic reaction in reaction chamber 12 (between enzyme 32 and substrate 30) causes formation of a product 34. Over time, this reduces the amount of substrate in reaction chamber 12 and causes substrate 30 to migrate from reservoir chamber 11 to reaction chamber 12. This results in less substrate in reservoir chamber 11. The amount of substrate in reservoir chamber 11 is thus indicative 35 of the reaction occurring in reaction chamber 12.

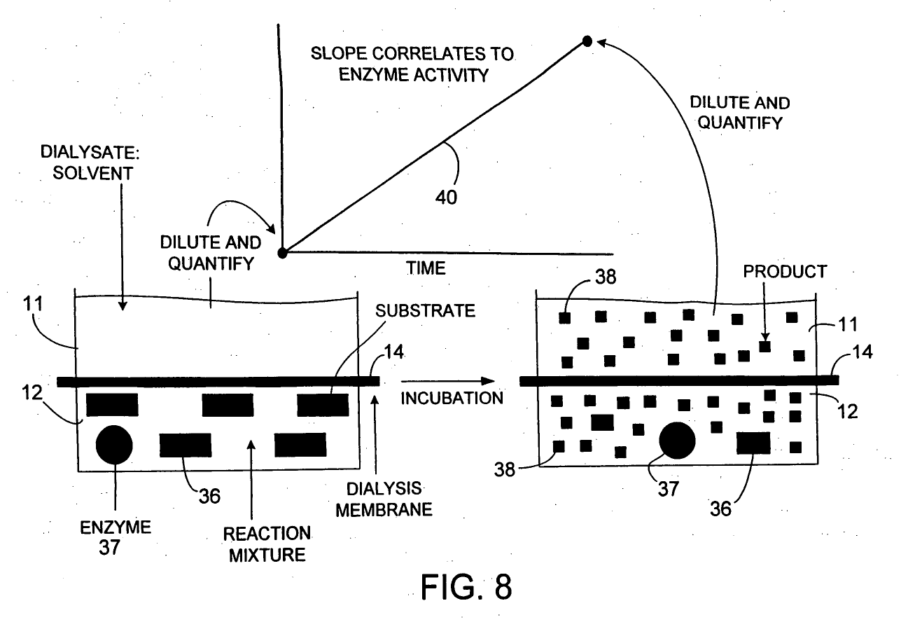

The assay described herein may also be used to measure the activity of any enzyme that breaks down a large substrate into a smaller product. Examples of such enzymes include, but are not limited to, Dnase, Rnase, any restriction endonuclease, protease, and amalase. FIG. 8 shows an illustration of how the assay works in this context. More specifically, initially, in this example, reservoir chamber 11 does not contain any substrate 38. A reaction in reaction chamber 12 (e.g., resulting from an enzyme 37 breaking-down the product 36) results in a substrate 38, which is able to pass through membrane 14. As the reaction progresses, more substrate is produced in reaction chamber 12. This substrate diffuses (migrates) to reservoir chamber 11. As above, larger molecules of product and enzyme are prevented from migrating to reservoir chamber 11. Thus, by detecting the amount of product in reservoir chamber 11, it is possible to detect the rate of the reaction 40 occurring in reaction chamber 12. The assay may be used to test the effect of a test substance on the reaction occurring in the reaction chamber, in the manner described above. For example, the assay may be used to test whether a test substance inhibits or promotes the reaction.

The assay described herein is not limited to the embodiments disclosed. The assay may be used to measure any nucleotide concentration without interference from other reaction components through use of a dialysis device. The device can be used with a variety of enzymatic reactions. The assay can performed by migrating the assay to capillary electrophoresis to increase throughput and increase sensitivity and accuracy. Either direct nucleotide measurement by UV absorbance or LIF (laser induced fluorescence) detection of labeled primers can be used to rank enzymatic (e.g., telomerase) inhibitor compounds for their ability to affect enzyme activity.

Elements of different embodiments described herein may be combined to form other embodiments not specifically set forth. Other embodiments not specifically described herein are also within the scope of the following claims.

1. A method comprising:
exposing an enzyme to a substrate and a test substance in a first chamber; and
detecting an effect on the substrate in a second chamber following exposure of the enzyme to the substrate and the test substance in the first chamber.

2. The method of claim 1, wherein the enzyme comprises telomerase and the substrate comprises nucleotides.

3. The method of claim 1, wherein detecting comprises determining a change in an amount of light absorbed by the substrate in the second chamber following exposure.

4. The method of claim 3, wherein the light comprises ultraviolet light.

5. The method of claim 1, wherein exposing the enzyme to the substrate in the first chamber causes a reaction that reduces an amount of free substrate in the first chamber; and
wherein the effect comprises the substrate in the second chamber migrating to the first chamber in response to a reduction in the amount of free substrate in the first chamber.

6. The method of claim 5, wherein the reaction comprises formation of single-stranded DNA using the substrate and the enzyme.

7. The method of claim 5, wherein the test substance affects the reaction.

8. The method of claim 7, wherein an effect on the reaction comprises one of inhibiting the reaction and promoting the reaction.

9. A method of measuring activity of an enzyme using a dialysis device comprised of a reaction chamber containing a substrate, a reservoir chamber containing the substrate, and a membrane separating the reaction chamber and the reservoir chamber, where the membrane allows passage of the substrate, the method comprising:
exposing the substrate in the reaction chamber to an enzyme and a test substance, the membrane inhibiting passage of the enzyme and the test substance; and
detecting a change in an amount of the substrate in the reservoir chamber after the substrate has been exposed to the enzyme and the test substance for a time, the amount of the substrate in the reservoir chamber corresponding to an amount of the substrate that has passed through the membrane from the reservoir chamber to the reaction chamber;
wherein a reaction that occurs in the reaction chamber between the substrate and the enzyme affects the amount of the substrate that passes through the membrane from the reservoir chamber to the reaction chamber; and
wherein the test substance affects the reaction.

10. The method of claim 9, wherein the test substance inhibits the reaction.

11. The method of claim 9, wherein the enzyme comprises at least one of: telomerase, DNA polymerase, RNA polymerase, RNA ligase, ribosomes, and starch synthase.

12. The method of claim 9, wherein the enzyme comprises telomerase, the substrate comprises nucleotides, and the test substance comprises a telomerase inhibitor.

13. The method of claim 1, wherein detecting the change in the amount of the substrate comprises measuring a change in absorption of ultraviolet light by the substrate in the reservoir chamber.

14. An apparatus comprising:
a reservoir chamber that contains a substrate;
a reaction chamber that contains the substrate, an enzyme, and a test substance; and
a dialysis membrane that separates the reservoir chamber and the reaction chamber, the dialysis membrane permitting passage of the substrate and inhibiting passage of the enzyme and the test substance;
wherein the test substance has an effect on a reaction between the substrate and the enzyme, the reaction influencing an amount of the substrate that migrates from the reservoir chamber, through the dialysis membrane, to the reaction chamber.

15. The apparatus of claim 14, wherein the test substance influences the reaction by inhibiting the reaction or promoting the reaction.

16. The apparatus of claim 14, wherein the enzyme comprises at least one of: telomerase, DNA polymerase, RNA polymerase, RNA ligase, ribosomes, and starch synthase.

17. The apparatus of claim 14, wherein the enzyme comprises telomerase, the substrate comprises nucleotides, and the test substance comprises a telomerase inhibitor.

18. The apparatus of claim 14, further comprising an ultraviolet light source that exposes at least the reservoir chamber to ultraviolet light;
wherein an amount of ultraviolet light absorbed by the substrate in the reservoir chamber corresponds to the effect the test substance has on the reaction.

19. The apparatus of claim 14, wherein the reaction comprises formation of single-stranded DNA using the substrate and the enzyme.

20. The apparatus of claim 14, wherein the reservoir chamber comprises a low volume equilibrium dialysis chamber.
